(19) United States
(12) Patent Application Publication
(10) Pub. No.: US 2006/0013899 A1
(43) Pub. Date: Jan. 19, 2006

(54) Topical compositions for treatment of skin disorders and methods of use thereof

(76) Inventor: Glen Lockhart, San Diego, CA (US)

Correspondence Address: T. D. FOSTER, 12760 HIGH BLUFF DRIVE, SUITE 300, SAN DIEGO, CA, 92130, US

(21) Appl. No.: 11/144,524

(22) Filed: Jun. 3, 2005

Related U.S. Application Data
(60) Provisional application No. 60/588,220, filed on Jul. 14, 2004.

(51) Int. Cl. A61K 36/82; A61K 36/28; A61K 31/555

(52) U.S. Cl. 424/729; 514/184; 424/757; 424/764

(57) ABSTRACT

The present invention generally relates to topical compositions, and particularly to a topical ointment for treatment of skin disorders and methods of use thereof The present invention recognizes that treatment of certain skin disorders can be more efficient, safe, and rapid-acting with few or no side effects. The present invention is particularly adaptable for treatment of a variety of skin disorders, such as for example, dermatitis (eczema), psoriasis and other forms of immuno-dysfunctional dermatitis. The present invention can be combined as supplement treatment to many available over-the counter and/or prescriptive products for treatment of skin disorders.

### FIELD OF INVENTION

The present invention relates generally to the field of topical compositions, and particularly to a topical ointment for treatment of skin disorders and methods of use thereof

### BACKGROUND OF THE INVENTION

Atopic dermatitis (eczema), psoriasis, and other forms of immuno-dysfunctional dermatitis affect approximately six to seven percent of the population in the USA. It is estimated eczema affects ten percent of the infant population and three percent of the total USA population. Psoriasis affects another two percent of the USA population. These conditions are acute or chronic noncontagious inflammations of the skin characterized by erythema (redness), edema (accumulation of watery fluid), oozing, itching, scaling, crusting, or hardened skin.

A variety of over-the counter and prescriptive products for treatment of these skin disorders are commercially available. However, many of the available treatments have limited success or success limited only to mild forms of the disorder. Furthermore, many of the available treatments have restricted use and may cause multiple detrimental side effects. The side effects from various prescriptive drugs may include bleeding, bruising, atrophy, hypopigmentation, telangiectasia, hypothalamic suppression, growth inhibition in children, reduced immunity development, lymphadenopathy, lower respiratory tract infection, gastroenteritis, staphylococcal infection, viral infections, tuberculosis, liver damage, fetal death and birth defects, high blood pressure, kidney disease, bone marrow suppression, and others.

While there has been a trend to produce more rapid-acting treatment products with fewer side effects, further improvements in the effectiveness and safety of a product that begins relieving symptoms and/or achieve remission quickly and causes little or no detrimental side effects are desirable, and the present invention addresses the existing problems and provides related benefits.

### SUMMAY OF THE INVENTION

The present invention is directed to topical compositions, and particularly to a topical ointment for treatment of skin disorders and methods of use thereof The present invention recognizes that treatment of certain skin disorders can be more efficient, safe, and rapid-acting with few or no side effects. The present invention is particularly adaptable for treatment of a variety of skin disorders, such as for example, dermatitis (eczema), psoriasis and other forms of immuno-dysfunctional dermatitis. The present invention can be combined as supplement treatment to many available over-the counter and/or prescriptive products for treatment of skin disorders.

One aspect of the present invention is a topical composition for treatment of skin disorders including zinc pyrithione, green tea (Camellia Oleifera and/or Camellia Sinensis), and hydrocotyl (Centella Asiatica) to produce synergistic beneficial effects. The composition of the present invention may also include any other beneficial ingredients, such as for example, essential fatty acid, lipids, aloe vera, betaglucan, soy, amino acids, adenosine triphosphate, panthenol, calcium, zinc, antioxidants, and tocopherol.

An object of the present invention is to provide for suitable compositions and methods for treatment of skin conditions associated with skin disorders including, but not limited to, eczema, psoriasis and other forms of immuno-dysfunctional dermatitis, such as for example, seborrheic dermatitis, nummular dermatitis, herpes zoster (shingles), rosacea, basal carcinomas, skin diseases involving pruritus, skin traumas such as burns, abrasions and cuts, slow healing skin injuries, and sunburn.

Another object of the present invention to provide for suitable compositions and methods for treatment, control, or remission of these conditions within short period of time, such as for example, a matter of two to five weeks.

Another object of the present invention is to provide for suitable compositions and methods for treatment, control, or remission of these conditions for a large percentage of affected individuals, such as for example, eighty to ninety percent of cases including moderate to severe cases.

Another object of the present invention is to provide for suitable compositions and methods for treatment, control, or remission of these conditions that has substantially no adverse side effects and safe for infants, adults, and those with other immuno-suppression conditions.

### DETAILED DESCRIPTION OF THE INVENTION

INTRODUCTION

The present invention recognizes that topical compositions for treatment of certain skin disorders can be more efficient, safe, and rapid-acting with few or no side effects. The present invention is particularly adaptable for treatment of a variety of skin disorders, such as for example, dermatitis (eczema), psoriasis and other forms of immuno-dysfunctional dermatitis.

As a non-limiting introduction to the breadth of the present invention, the present invention includes several general and useful aspects, including:
1) A topical composition for treatment of skin disorders including zinc pyrithione, green tea (Camellia Oleifera and/or Camellia Sinensis), and hydrocotyl (Centella Asiatica) to produce synergistic beneficial effects.
2) A method for treatment of skin disorders including providing a topical composition including zinc pyrithione, green tea (Camellia Oleifera and/or Camellia Sinensis), and hydrocotyl (Centella Asiatica); and applying the topical composition to affected areas of the skin to produce synergistic beneficial effects.

Further objectives and advantages of the present invention will become apparent as the description proceeds. To gain a full appreciation of the scope of the present invention, it will be further recognized that various aspects of the present invention can be combined to make desirable embodiments of the invention.

Unless defined otherwise, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art.

TOPICAL COMPOSITIONS AND METHODS FOR TREATMENT OF SKIN DISORDERS

The present invention is generally related to topical compositions, and particularly to a topical ointment for treatment of skin disorders and methods of use thereof The present invention is particularly adaptable for treatment of a variety of skin disorders, such as for example, dermatitis (eczema), psoriasis and other forms of immuno-dysfunctional dermatitis.

Skin scrapings and punch biopsies taken from eczema and psoriasis lesions reveal disproportionately high concentrations of histamine and the cytokines interleukin-1, interleukin-6, interleukin-8, tumor necrosis factor-alpha and interferon-gamma. These five cytokines plus histamine are powerful pro-inflammatory agents and these high concentration levels are capable of maintaining the hyper-inflammatory conditions consistent with eczema, psoriasis and other forms of inflammatory dermatitis.

The present invention includes zinc pyrithione, which has been used in other preparations for various types of skin conditions. Zinc pyrithione may be used to control mild to possibly moderate eczema and psoriasis. However, zinc pyrithione by itself fails to control these diseases in the moderate to severe ranges.

With the assistance of test patients it was determined that when zinc pyrithione, suspended in a water solution, was applied to mild to moderate eczema lesions it relieved the itching sensation within fifteen to thirty minutes and continued to provide this relief for up to six hours. It was also observed that during this period erythema was reduced. Anecdotally, zinc pyrithione appears to produce a transient histamine blocking or inhibiting mechanism. There is limited published laboratory research on zinc pyrithione and no materials could be found supporting this observation. Clinical data was published on the various benefits of zinc pyrithione contained in the product SkinCap™, however, it was later revealed this product also contains the steroid clobetasol propionate which is known to produce these various benefits. Later clinical testing of 24 patients treated with a solution of clobetasol propionate and a solution of clobetasol propionate and zinc pyrithione determined zinc pyrithione did not appear to enhance the efficacy of clobetasol propionate. The Use of 0.25% Zinc Pyrithione Spray Does Not Enhance the Efficacy of Clobetasol Propionate: Journal of the American Academy of Dermatology; 49(1):79-82, (July 2003).

Zinc pyrithione provides antibacterial and antifungal control. This may reduce infections and thereby reduce secondary inflammation caused by infection.

There is little published research on zinc pyrithione and no research dealing with modulation of cytokine activities or production. Cutaneous inflammation promotes the production of additional pro-inflammatory cytokines. It is possible that the inhibiting affect of zinc pyrithione on histamines may down-regulate pro-inflammatory cytokines to a degree, however, not substantially enough to control moderate to severe eczema or psoriasis.

The content of the present invention may contain any suitable amount of zinc pyrithione, preferably between about 0.1% to about 5.0% and more preferably between about 0.1% to about 1.0% and preferably between about 0.2% to about 0.5% by weight.

The present invention includes green tea (Camellia Oleifera and/or Camellia Sinensis). Inflammatory cascades increase oxidative stress levels within the skin, promoting an H₂O₂ mediated phosphorylation of mitogen-activated protein kinase signaling (PKC enzyme signaling). The normal twenty-eight day cutaneous cell cycle (from basal cellular division to exfoliation) can escalate to a three to six day cycle generating volumes of free radicals and subsequent inflammation, plus flaky, scaly, and hardened skin lesions. Green tea (epigallocatechin-3-gallate contained in green tea) treatment of the skin inhibits H₂O₂ production to reduce rapid oxidative stress-induced hyperkeratinization. This reduces the flaky, scaly, and hardening lesions of the skin. Topical treatment of green tea results in prevention of inflammatory responses, immunosuppression and oxidative stress, which are the biomarkers of skin diseases, such as eczema and psoriasis. Inhibition of UVB-Induced Oxidative Stress-Mediated Phosphorylation of Mitogen-Activated Protein Kinase Signalin: Toxicology and Applied Pharmacology, 176(2):110-7 (October 2001).

Green tea and epigallocatechin-3-gallate are also potent inhibitors of interleukin-8 releases, caused by tumor necrosis factor-alpha mediated expression of the interleukin-8 gene. Epigallocatechin-3-gallate inhibits tumor necrosis factor-alpha mediated interleukin-8 expression in a dose related manner. Among the green tea catechins, epigallocatechin-3-gallate is the most effective in reducing interleukin-8 production. Comparative Effects of Polyphenols from Green Tea: Achives of Dermatological Research, 295(3):112-6 (July 2003); A Green Tea-Derived Polyphenol: Inflammation, 26(5):233-41 (October 2002); Mechanisms of Cancer Prevention by Tea Polyphenols Based on Inhibition of TNF-alpha Expression, BioFactors, 1391(4):67-72 (2000).

Green tea has an inhibitory effect on tumor necrosis factor-alpha gene expression mediated through inhibition of nuclear factor-kappa B and activator protein-1. Oral or topical treatment with green tea reduces tumor necrosis factor-alpha gene expression as well as tumor necrosis factor-alpha protein levels and thereby reduces interleukin-1 and interleukin-10 gene expression. A New Function of green Tea: Prevention of Lifestyle-Related Diseases, Annals of the New York Academy of Sciences, 928:274-80 (April 2001).

Green tea catechin (epigallocatechin-3-gallate) has a number of unique antimicrobiological activities such as antibacterial, antiviral, antifungal and antitoxic effects. It exhibits lethal toxicity of staphylococcal enterotoxin B-induced production of tumor necrosis factor-alpha, interferon-gamma and interleukin-4. Inhibitory Effect of Catechin Against the Superantigen Staphylococcal Exterotoxin B, Archives of Dermatological Research, 295(5):183-9 (September 2003).

Tea suppresses type I and type IV allergic reactions. 118 test patients with moderate to severe eczema drank oolong tea (black) three times daily for thirty days and 74 (63%) showed marked to moderate improvement in their condition. A Trial of Oolong Tea in the Management of Recalcitrant Atopic Dermatitis, Archives of Dennatology, 137(1):42-43 (January 2001).

The content of the present invention may contain any suitable amount of green tea, preferably between about 0.5% to about 12.0% and more preferably between about 1% to about 6.0% and more preferably between about 1.5% to about 3.5% by weight.

Triterpenes, including madecassoside, contained in hydrocotyl (Centella Asiatica) have anti-inflammatory and regulatory effects on cutaneous keratinocytes. The T-lymphocytes, activated by inflammatory cascades, release interferon-gamma, which stimulates neuromediators by keratinocytes, leading to hyperproliferation of the T-lymphocytes. This provokes increased production of interferon-gamma and protease levels, which in turn causes more synthesis of interleukin-1 and interleukin-8. These cytokines participate in maintaining a pro-inflammatory cycle, such as seen with eczema and psoriasis. Madecassoside prevents and corrects immune dysfunction by reducing the synthesis of the main pro-inflammatory cytokines, including interleukin-1, interleukin-8 and interferon-gamma. Innovative Natural Active Ingredient with Anti-Inflammatory Properties, Cosmetics & Toiletries, 118(11):63-68 (November 2003).

Hydrocotyl has effective antioxidant properties to reduce free radical and inflammatory stress. Anecdotally, it has been said to have wound healing properties. Nutritional Support for Wound Healing: Alternative Medicine Review, 8(4):359-77 (November 2003).

The content of the present invention may contain any suitable amount of hydrocotyl, preferably between about 0.5% to about 12.0% and more preferably between about 1.0% to about 5.0% and more preferably between about 2.0% to about 3.5% by weight.

Cytokines have intricate relationships and complex balances which science is only beginning to understand. Very specific chemical conditions must be in place to modulate gene expression and there appears to be checks and balances that make these chemical processes even more tenuous and complex.

Individually zinc pyrithione provides some degree of control benefits for mild and possibly moderate eczema, psoriasis and other forms of immuno-dysfunctional dermatitis. Green tea and hydrocotyl individually show little visible benefits in the control of these diseases. The combination of these three ingredients, however, produces unexpected and remarkable benefits. The explanations above provide some validation of how these three ingredients may provide limited benefits, but cannot explain the dynamics of their relationship in the control of pro-inflammatory cytokines and the speed of the repair and healing process.

Zinc pyrithione is approved by the FDA for over-the-counter drug use at concentrations up to 0.25%. It has no known side effects other than normal allergic reaction in less than one percent of the population. Green tea and hydrocotyl are approved for over-the-counter and cosmetic use. They have no known side effects other than normal allergic reaction in less than one percent of the population. The present invention has no known side effects other than allergic reaction as would be possible with any skin care product.

Compositions for the present invention might include, but not limited to, lotions, creams, gels, solutions, oils, sticks, sprays, ointments, balms, shampoos, and pastes in an emulsion of the oil-in-water or water-in-oil type. Formulated in these manners, the present composition may contain any suitable amount of the ingredients, preferable the composition would contain between about 1.1% to about 29% and more preferably between about 2.1% to about 12.0% and more preferably between about 3.7% to about 7.5% by weight of the ingredients described above. This composition could also contain any other beneficial ingredients, such as for example, agents for hydration and moisturization, essential fatty acids, lipids, aloe vera (Barbadensis) gel, betaglucan, soy, amino acids, adenosine triphosphate, panthenol, calcium, zinc, antioxidants, and tocopherol.

### EXAMPLES

### Example I

### Topical Composition and Method for Treatment of Skin Disorders

Seventy-nine people who claimed to have been diagnosed with eczema by a physician used the present invention as per the regimen prescribed. They received an amount able to treat eighteen square inches of skin or less as per the prescribed regimen. Three people achieved no improvement. Seventy-six people achieved satisfactory to excellent results and most observed full remission. Ten people required an additional amount of the product because the affected area was presumably larger than eighteen square inches. One patient voluntarily wrote, “Three weeks of use (of the invention) did more for my eczema than three years of monthly visits to the dermatologist.”

All headings are for the convenience of the reader and should not be used to limit the meaning of the text that follows the heading, unless so specified. Various changes and departures may be made to the present invention without departing from the spirit and scope thereof Accordingly, it is not intended that the invention be limited to that specifically described in the specification, but only as set forth in the claims. All listed publications are incorporated herein by reference in their entirety.

What is claimed is:

1. A topical composition for treatment of skin disorders comprising:
zinc pyrithione;
green tea; and
hydrocotyl
wherein, said topical composition produces synergistic beneficial effects for said treatment of skin disorder.

2. The topical composition of claim 1, wherein the amount of zinc pyrithione comprises between about 0.1% to about 5.0% by weight, the amount of green tea comprises between about 0.5% to about 12.0% by weight, and the amount of hydrocotyl comprises between about 0.5% to about 12.0% by weight.

3. The topical composition of claim 1, wherein the amount of zinc pyrithione comprises between about 0.2% to about 0.5% by weight, the amount of green tea comprises between about 1.5% to about 3.5% by weight, and the amount of hydrocotyl comprises between about 2% to about 3.5% by weight.

4. The topical composition of claim 1, further comprising one or more of essential fatty acids, lipids, aloe vera, betaglucan, soy, amino acids, adenosine triphosphate, panthenol, calcium, zinc, antioxidants, and tocopherol.

5. The topical composition of claim 1, wherein said green tea comprises Camellia Oleifera or Camellia Sinensis or both.

6. The topical composition of claim 1, wherein said hydrocotyol comprises Centella Asiatica.

7. The topical composition of claim 1, wherein said skin disorder comprises one or more of eczema, psoriasis and other forms of immuno-dysfunctional dermatitis, such as for example, seborrheic dermatitis, nummular dermatitis, herpes zoster (shingles), rosacea, basal carcinomas, skin diseases involving pruritus, skin traumas such as burns, abrasions and cuts, slow healing skin injuries, and sunburn.

8. A method for treatment of skin disorders for a patient comprising the steps of:
a) administering a topical composition comprising zinc pyrithione, green tea, and hydrocotyl; and
b) applying the topical composition to affected areas of the skin to produce synergistic beneficial effects.

9. The method of claim 8, wherein the amount of zinc pyrithione comprises between about 0.1% to about 5.0% by weight, the amount of green tea comprises between about 0.5% to about 12.0% by weight, and the amount of hydrocotyl comprises between about 0.5% to about 12.0% by weight.

10. The method of claim 8, wherein the amount of zinc pyrithione comprises between about 0.2% to about 0.5% by weight, the amount of green tea comprises between about 1.5% to about 3.5% by weight, and the amount of hydrocotyl comprises between about 2% to about 3.5% by weight.

11. The method of claim 8, further comprising one or more of essential fatty acids, lipids, aloe vera, betaglucan, soy, amino acids, adenosine triphosphate, panthenol, calcium, zinc, antioxidants, and tocopherol.

12. The method of claim 8, wherein said green tea comprises Camellia Oleifera or Camellia Sinensis or both.

13. The method of claim 8, wherein said hydrocotyol comprises Centella Asiatica.

14. The method of claim 8, wherein said skin disorder comprises one or more of eczema, psoriasis and other forms of immuno-dysfunctional dermatitis, such as for example, seborrheic dermatitis, nummular dermatitis, herpes zoster (shingles), rosacea, basal carcinomas, skin diseases involving pruritus, skin traumas such as bums, abrasions and cuts, slow healing skin injuries, and sunburn.
